(19) United States
(12) Patent Application Publication
(10) Pub. No.: US 2013/0172901 A1
(43) Pub. Date: Jul. 4, 2013

(54) TOOL FOR INSERTING FINE TUBULAR OBJECTS INTO THE COCHLEA

(71) Applicants: Alexis Bozorg Grayeli, Paris (FR); Olivier Sterkers, Paris (FR); Mathieu Miroir, Paris (FR); Yann Nguyen, Paris (FR); Evelyne Ferrary, Paris (FR)

(72) Inventors: Alexis Bozorg Grayeli, Paris (FR); Olivier Sterkers, Paris (FR); Mathieu Miroir, Paris (FR); Yann Nguyen, Paris (FR); Evelyne Ferrary, Paris (FR)

(73) Assignee: Institut National de la Sante et de la Recherche Medicale (INSERM), Paris (FR)

(21) Appl. No.: 13/810,740

(22) PCT Filed: Jul. 13, 2011
(86) PCT No.: PCT/FR2011/051691

§ 371 Date: Mar. 19, 2013

(30) Foreign Application Priority Data
Jul. 19, 2010 (FR) 10 55870

(51) Int. Cl. A61F 2/18

(52) U.S. Cl. CPC A61F 2/18
USPC 606/108

(57) ABSTRACT

A tool for inserting fine tubular objects into the cochlea is provided. The tool has a gripping body, which forms a handle and ends in a tubular guide for the fine tubular object, and a pusher engaged in the tubular guide in order to push the fine tubular object out of the guide and into the cochlea. The tool is equipped with: an electronic control unit; motorized means controlled by the control unit in order to move the pusher; a force-measuring means connected to the control unit and arranged so as to be sensitive to a resistance force experienced by the fine tubular object during insertion into the cochlea and supplying a signal that can be exploited in order to estimate the resistance force; and means for indicating the resistance force connected to the control unit.

The present invention relates to a tool for inserting fine tubular objects into the cochlea, for example a cochlear implant of a hearing aid, or else a catheter.

### BACKGROUND ART

Hearing aids are known that have an extracranial speech processor capturing the external sounds and converting them into a sequence of electrical signals that are sent to a transmitter so as to be output in the direction of an intracranial receiver connected to a cochlear implant, which is arranged directly in the cochlea in such a way that it excites the receptors of the auditory nerve in response to the reception of the signals. The cochlear implant has the shape of a fine tube, which is engaged in the cochlea in such a way that the tube extends along substantially the entire length of the curvilinear course thereof.

The introduction and placement of the cochlear implant in the cochlea is a particularly delicate operation. This is because the cochlear implant is generally fitted in place under general anesthesia and with visual monitoring beyond the point of insertion. The way in which the cochlear implant positions itself in the cochlea is impossible to verify from the outside when fitting the cochlear implant, and it is necessary to wait for the control scan in order to verify whether the cochlear implant has been correctly positioned. If the cochlear implant is not correctly positioned, it is then necessary to withdraw the latter and fit another one in place, which requires general anesthesia again.

In order to help the practitioner, a tool has been developed for inserting fine tubular objects into the cochlea, said tool having a gripping body ending in a tubular guide in which the cochlear implant is placed. The tool has a fine pusher which slides inside the tubular guide in order to push the cochlear implant out of the guide and insert the cochlear implant into the cochlea. The pusher is actuated by an index which is mounted slidably on the body of the tool and which is pushed by one of the fingers of the hand that is holding the tool.

A tool of this type is described in the document WO 03/070133.

This type of tool has a number of disadvantages. Firstly, the internal friction between the index and the body far exceeds the resistance force of insertion of the cochlear implant into the cochlea, which force is very low, such that it is impossible for the practitioner to sense any unusual resistance during said insertion, which could indicate problematic insertion. The practitioner thus pushes the cochlear implant without having any idea of the resistance encountered by the cochlear implant during the insertion thereof. Moreover, the fact that the practitioner pushes the index with a finger of the hand that is holding the tool makes it awkward to hold the tool in position and can give rise to jerks during the insertion of the cochlear implant.

### PROBLEM ADDRESSED BY THE INVENTION

The problem addressed by the invention is to make available a tool for inserting fine tubular objects into the cochlea, without the disadvantages mentioned above.

### BRIEF DESCRIPTION OF THE INVENTION

With a view to solving this problem, the invention proposes a tool for inserting fine tubular objects into the cochlea, having a gripping body, which forms a handle designed to be held in a hand by a user, said gripping body ending in a tubular guide in which the fine tubular object is to be arranged, the inserting tool having a pusher engaged in the tubular guide in order to push the fine tubular object out of the guide and insert it into the cochlea. The inserting tool is equipped with:
an electronic control unit,
motorized means placed in the gripping body and controlled by the control unit in order to move the pusher,
force-measuring means placed in the gripping body, connected to the control unit and arranged so as to be sensitive to a resistance force experienced by the fine tubular object during the insertion thereof into the cochlea and supplying a signal that can be exploited in order to estimate said resistance force,
means for indicating said resistance force placed on the gripping body and connected to the control unit.

Thus, the inserting tool, which can be carried by hand, comprises motorized means that can be actuated automatically by the control unit, for example by simple pressure applied to a switch, which facilitates the handling of the tool. The use of motorized means permits an insertion at substantially constant speed, which eliminates any risk of jerks. Finally, the indication of the resistance force during insertion allows the practitioner to follow this resistance force in real time. This information permits detection of several events:
a final resistance, which indicates that the fine tubular object has reached the end of the cochlea; the motorized means can then be controlled to automatically stop the advance in response to the detection of a defined force threshold;
a resistance during the course of insertion, which can signify that the fine tubular object is poorly positioned or that the cochlea is obstructed; in this case, the practitioner, duly informed of this resistance, can choose to interrupt the operation and, if appropriate, begin it again with another fine tubular object, without waiting for the patient to wake and without waiting to see the control scan.

### BRIEF DESCRIPTION OF THE FIGURES

The invention will be better understood in light of the following description of particular embodiments and by reference to the figures of the attached drawings, in which:

FIG. 1 is a perspective view of a tool for helping insert fine tubular objects into the cochlea, according to a first particular embodiment of the invention;

FIG. 2 is a partial sectional view of the tool from FIG. 1;

FIG. 3 is a perspective view of a tool for helping insert fine tubular objects into the cochlea, according to a second particular embodiment of the invention;

FIG. 4 is a partial sectional view of the tool from FIG. 3;

FIG. 5 is a detail according to the arrow V in FIG. 4, the body having been omitted;

FIG. 6 is a graph of the force of inserting a cochlear implant into a cochlea as a function of the movement of the pusher.

### DETAILED DESCRIPTION OF THE INVENTION

Referring to FIG. 1, and in accordance with a first particular embodiment of the invention, the inserting tool comprises a gripping body 1 forming a handle that can be held in a hand by the practitioner or fitted to the end of a surgical robot. The inserting tool comprises a tubular guide 2 with a proximal portion, which is fixed to one of the ends of the gripping body 1, and a distal portion, which has a free end and in this case forms an angle with the proximal portion. The guide 2 receives a cochlear implant 200, of which the connecting wire 201 emerges from the guide 2 through a longitudinal slit 3 of the latter. The guide 2 is introduced through the external auditory meatus after detachment of the skin and of the tympanum, or else by mastoidectomy and tympanotomy, such that its free end comes to lie opposite an orifice made in the wall of the posterior cochlea (cochleostomy or round window of the cochlea) in order to introduce the cochlear implant 200 there.

In this embodiment, the inserting tool is equipped with an electronic control unit which comprises a control switch 4 of the inserting tool, supported by the gripping body, and a screen 5 providing the practitioner with information, particularly concerning the resistance force experienced by the cochlear implant 200 during the insertion thereof into the cochlea, as will be explained in detail below.

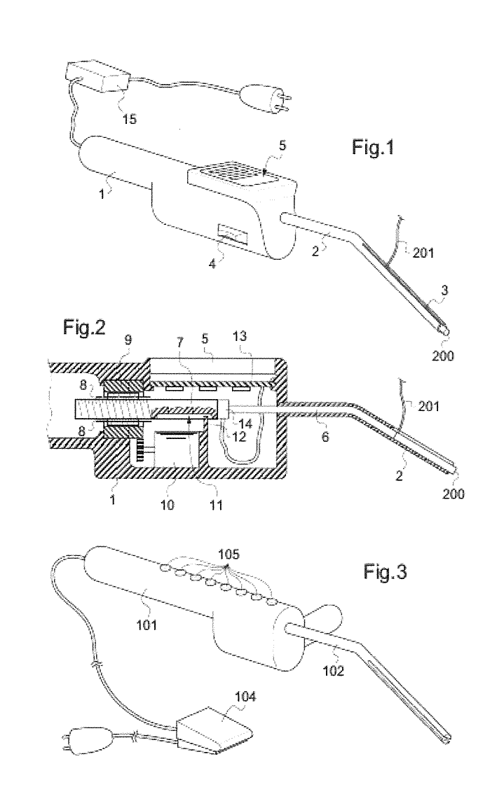

Referring to FIG. 2, the inserting tool comprises a pusher 6 introduced into the guide 2 in order to push the cochlear implant 200 inside the cochlea. At least the end part of the pusher 6 is flexible in order to follow the angle of the guide 2. The pusher is itself pushed by a piston 7 mounted slidably in the gripping body 1 in an axis of movement that coincides with a longitudinal axis of the proximal portion of the guide 2. The piston 7 has a threaded part which cooperates with threaded rollers 8, which themselves cooperate with a ring 9 mounted rotatably on the gripping body 1, inside the latter, such that a rotation of the ring 9 causes a longitudinal movement of the piston 7 and, as a result, a longitudinal movement of the pusher 6.

The rotation of the ring 9 is brought about by means of an electric motor 10 placed in the gripping body 1 and controlled by the control unit to drive the ring 9 by way of a pinion that cooperates with an outer toothing of the ring 9. To prevent the piston 7 from turning, it has a longitudinal groove 11 in which is engaged a finger 12 integral with the gripping body 1.

The switch 4 of the control unit is a three-position switch, one of which positions is a stable intermediate position to which the switch is returned in the absence of pressure and in which the electric motor 10 is not powered. In the two other positions, the electric motor is powered so as to turn in one direction or the other.

The control unit also comprises an electronic card 13 which is contained in the gripping body 1 and which bears electronic components that manage the powering of the electric motor 10 and also the screen 5.

Here, the supply of electricity is provided by a transformer 15 which is connected to the mains. Placing the transformer outside the gripping body 1 has the effect that the tool is lighter, making it easier to handle during insertion of the cochlear implant.

According to an essential aspect of the invention, a force sensor 14, here a sensor of the piezoelectric type, is interposed between the piston 7 and the pusher 6 in order to measure a resistance force experienced by the pusher 6.

The resistance force experienced by the pusher 6 is the sum of a frictional force between the guide and the pusher and a resistance force experienced by the cochlear implant during its insertion into the cochlea. The aforementioned frictional force is essentially constant (except briefly when the electric motor 10 is started) in the sense that the electric motor 10 turns at constant speed. It is easy to calibrate this resistance force in order to deduct it from the force measured by the force sensor 14, by which means it is possible to obtain a realistic estimate of the resistance force experienced by the cochlear implant during its insertion into the cochlea.

The value of this resistance force is permanently displayed by the screen 5, for example in the form of a graphic scale, such that the practitioner can be informed of this value in real time. If the resistance force rises abnormally in the course of insertion, it is certainly the case that the cochlear implant is poorly positioned in the cochlea. The practitioner is thus immediately made aware of the problem.

In addition, at the end of its travel, the cochlear implant comes into abutment against the base of the cochlea, and the resistance force that is displayed rises sharply. The practitioner is thus informed that the operation is completed and he can release the switch. Alternatively, the control of the electric motor 10 can be arranged so as to stop the electric motor once the resistance force reaches a defined threshold.

The inventors have carried out various measurements allowing them to establish that the resistance force experienced by the cochlear implant during its insertion into the cochlea has the general form illustrated by the curve in FIG. 6, in which the values are given only by way of example and may vary from one patient to another. This curve has a first, relatively constant force plateau, and then, toward the end of the insertion, a transitory peak followed by a relaxation, before rising sharply when the cochlear implant reaches the base of the cochlea. By choosing a threshold S that is higher than the transitory peak, but that is capable of being reached during the final rise in the resistance force, it is possible to ensure that the motor is indeed stopped at the end of the insertion.

According to a first variant, the threshold is predetermined once and for all and is valid for all patients. According to a second variant, the control unit incorporates an algorithm that recognizes the form of the resistance-force curve in real time and locates the transitory peak followed by the relaxation. If such relaxation does not occur, this is because the cochlear implant is poorly positioned. The motor is therefore immediately stopped when the force reaches a predetermined threshold, and a corresponding message is displayed on the screen 5. If such relaxation does occur, this is because the insertion of the cochlear implant is proceeding normally. The algorithm then calculates a suitable force threshold, for example equal to 1.5 times the value of the transitory peak, which threshold will serve to stop the electric motor 10 at the end of insertion.

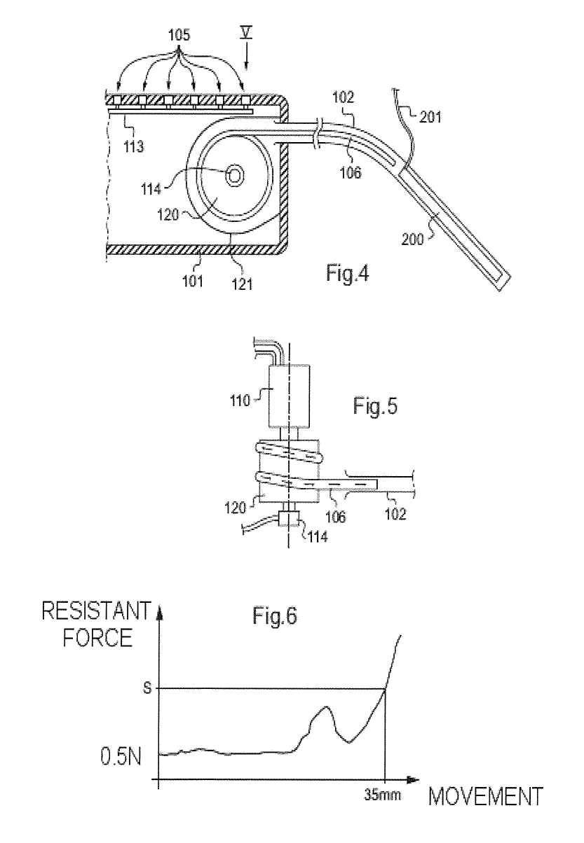

According now to a second embodiment illustrated in FIGS. 3 to 5, in which the reference numbers for the elements in common with FIGS. 1 and 2 are increased by a hundred, the inserting tool of the invention, which is portable just as in the first embodiment, comprises a gripping body 101 with a guide 102. The pusher 106 has its distal end engaged in the guide 102 and is wound onto a drum 120 which is mounted rotatably on the gripping body 101 about a transverse axis of rotation and is here driven directly by the electric motor 110 extending along the axis of rotation of the drum 120. In FIG. 3, the motor 110 can be seen protruding from the side of the body 101, so as to form a gripping handle that makes the tool easier to hold in the hand. A casing 121 surrounding the drum 120 constrains the pusher 106 so as to force the latter into the guide 102 as it is unwound.

The force sensor 114 is here a torque sensor, which measures the resistance torque experienced by the drum 120 during the rotation thereof. The torque sensor 114 measures a torque proportional to the sum of the frictional force experienced by the pusher 106 and the resistance force experienced by the cochlear implant 200 during its insertion into the cochlea. Since the frictional force is substantially constant, it suffices to subtract it from the measurement given by the torque sensor 114 in order to obtain an estimate of the resistance force experienced by the cochlear implant during its insertion.

In this particular embodiment, the resistance force is indicated by means of a series of LEDs 105 which light up one after another as the resistance force experienced by the cochlear implant increases during the insertion thereof into the cochlea.

Here, the switch of the preceding embodiment has been replaced by a pedal 104 controlling the rotation of the motor. Thus, the practitioner's hands are used only to hold the tool.

The pedal 104 here encloses the transformer 115, of which the power cord can be seen extending as far as a plug for connecting the tool to the mains. The tool is therefore very light and easy to keep in place.

Of course, the invention is not limited to what has just been described and instead encompasses any variants covered by the scope defined by the claims.

In particular, although the motor and the indicating means have been described as being powered by an external transformer, it is of course also possible to equip the tool with batteries accommodated in the body of the tool.

Although the indicating means have been described as comprising a screen or a series of LEDs, the resistance force experienced by the cochlear implant during its insertion will of course be able to be indicated by any other means, for example by an acoustic device producing a sound whose intensity or pitch depends on the value of the resistance force.

Although the tool has been described as being used to insert a cochlear implant, it will also be possible to use the tool to insert any other fine tubular object, for example a catheter for delivering treatment directly inside the cochlea.

Although it has been stated that the practitioner holds the tool by its body, it will be possible for the body of the tool to be fitted to the end of a surgical robot or to the plate of a fixed support holding the tool steady during insertion.

Although the motorized means have been described as comprising an electric motor, it will be possible to use other motorized means, such as a pneumatic or hydraulic motor.

Although the tool has been described as being equipped with force-measuring means in the form of a force sensor inserted in the transmission chain between the electric motor and the pusher so as to be sensitive to the resistance force experienced by the cochlear implant during the insertion thereof into the cochlea, it will be possible for the force sensor to be placed at locations other than those illustrated, for example at the end of the pusher, in order to measure directly the resistance force experienced by the object that is pushed by the pusher. It will also be possible to use means other than a force sensor, for example means for measuring the current flowing in the coils of the motor, in order to estimate the torque generated by the latter.

Finally, it will be possible to equip the tool with other measuring means, for example a gyroscope for detecting any movement from the nominal direction of the distal portion of the guide during insertion. Preferably, the gyroscope will then be coupled to indicating means for showing the practitioner in which direction the tool has to be adjusted in order to recover the nominal direction of the distal portion of the guide during insertion.

In addition, it will be possible to equip the tool with a fiber optic running along the guide, in order to allow the practitioner to see the interior of the patient's inner ear during the insertion operation.

1. A tool for inserting fine tubular objects into the cochlea, having a gripping body, which forms a handle designed to be held in a hand by a user, said gripping body ending in a tubular guide in which the fine tubular object is to be arranged, the inserting tool having a pusher engaged in the tubular guide in order to push the fine tubular object out of the guide and insert it into the cochlea,
wherein said inserting tool is equipped with:
an electronic control unit,
motorized means placed in the gripping body and controlled by the control unit in order to move the pusher,
force-measuring means placed in the gripping body, connected to the control unit and arranged so as to be sensitive to a resistance force experienced by the fine tubular object during the insertion thereof into the cochlea and supplying a signal that can be exploited in order to estimate said resistance force,
means for indicating said resistance force placed on the gripping body and connected to the control unit.

2. The inserting tool as claimed in claim 1, in which the force-measuring means comprise a force sensor arranged between the pusher, which is introduced into the guide in order to push the fine tubular object, and a piston actuated by the motorized means.

3. The inserting tool as claimed in claim 1, in which the force-measuring means comprise a torque sensor arranged to measure a torque experienced by a drum which is mounted rotatably in the body, so as to be driven in rotation by the motorized means, and on which the pusher is wound.

4. The inserting tool as claimed in claim 1, in which the indicating means comprise a display screen arranged directly on the body of the tool.

5. The inserting tool as claimed in claim 1, in which the indicating means comprise a series of LEDs which are arranged directly on the body of the tool and which light up one after another as the resistance force experienced by the cochlear implant increases during the insertion thereof into the cochlea.

6. The inserting tool as claimed in claim 1, in which the motorized means are controlled to interrupt an advance of the pusher when the resistance force exceeds a given threshold.

7. The inserting tool as claimed in claim 6, in which the threshold is predetermined.

8. The inserting tool as claimed in claim 6, in which the threshold is fixed according to a value of a transitory peak of the resistance force detected during the insertion of the fine tubular object into the cochlea.
